(19) United States
(12) Patent Application Publication
(10) Pub. No.: US 2024/0122949 A1
(43) Pub. Date: Apr. 18, 2024

(54) METHOD OF TREATING VARIOUS DISORDERS AND CONDITIONS USING CITICOLINE

(71) Applicants: P S. John, Kottayam (IN); Varghese Thomas, Kottayam (IN); Ann Maria John, Kottayam (IN)

(72) Inventors: P S. John, Kottayam (IN); Varghese Thomas, Kottayam (IN); Ann Maria John, Kottayam (IN)

(21) Appl. No.: 18/079,067

(22) Filed: Dec. 12, 2022

(30) Foreign Application Priority Data
Oct. 14, 2022 (IN) 202241058727

(51) Int. Cl. A61K 31/6615; A61K 9/00; A61K 31/375; A61K 45/06; A61P 19/08

(52) U.S. Cl. CPC A61K 31/6615; A61K 9/0014; A61K 9/0056; A61K 31/375; A61K 45/06; A61P 19/08

(57) ABSTRACT

A method of treating or preventing various bone-related diseases and disorders in a human using an effective amount of citicoline with or without other nutraceutical agents is disclosed. The citicoline (Cytidine Diphosphate choline or Cytidine 5′-diphosphocholine) is administrated to the patient or human in need of such treatment to prevent osteoporosis, promote healing in delayed union, non-union, infected non-union of fracture, acceleration of osteoporotic fracture healing and spinal cord regeneration in spinal cord injury in combination with bone marrow aspirate. The effective amount of citicoline is administered topically, orally or injected into the human body to promote tissue repair. Further, the method of treating patients using an effective amount of citicoline with or without other nutraceutical agents accelerates fracture healing.

### CROSS-REFERENCE TO RELATED APPLICATION

The present Application for Patent claims priority to Indian patent Application No. 202241058727 entitled “METHOD OF TREATING VARIOUS DISORDERS AND CONDITIONS USING CITICOLINE” filed 14 Oct. 2022, which is hereby expressly incorporated by reference herein.

### TECHNICAL FIELD

The present invention relates to a method of treating patients with spinal cord injury. The present invention also relates to a method of treating or preventing a patient or human with a variety of bone-related diseases and other disorders and conditions using citicoline.

### BACKGROUND

Citicoline (cytidine diphosphate-choline or cytidine 5′-diphosphocholine) is a brain chemical that occurs naturally in the human body and is found in the cell membrane. It is beneficial in disorders of the brain that affect functions such as movement, memory, and mood changes, as it prevents injury to brain cells. It is popularly known as a brain tonic.

Citicoline is a nutraceutical used all over the world for the treatment of various neurodegenerative diseases including head trauma, cerebrovascular diseases, Alzheimer's disease, stroke, Parkinson's disease, various ophthalmic conditions, and as a memory enhancer. This drug is considered to be a very safe medication with negligible side effects. It is available as tablets, injections, and syrup and is considered a food supplement.

There exists various method for treating a variety of bone-related diseases and other conditions. Few existing patent references attempted to address the problems cited in the background as prior art over the presently disclosed subject matter and are explained as follows:

A prior art U.S. Pat. No. 5,583,117 A assigned to Reid von Borstel et. al., entitled “Acylated uridine and cytidine for elevating tissue uridine and cytidine” discloses a general method for delivering exogenous cytidine or uridine to the tissue of an animal comprising the administration of acylated cytidine or acylated uridine, respectively. Methods of treating myocardial infarction and cardiac insufficiency comprising the administration of acylated cytidine or acylated uridine, are also disclosed.

Another prior art WO 2005086619 A2 assigned to Perry F. Renshaw, et. al, entitled “Methods of treating psychiatric, substance abuse, and other disorders using combinations containing omega-3 fatty acids” discloses a method for treating or preventing psychiatric disorders, substance abuse disorders, and other conditions, e.g., cardiovascular disease and cancer, involving administration of a therapeutically-effective amount of a cytosine-containing or cytidine-containing compound, creatine-containing compound, adenosine-containing, adenosine-elevating compound, omega-3 fatty acids, or combinations thereof to a mammal. The invention further provides methods of enhancing neurodevelopment and delaying premature pregnancy by administration of an effective amount of a cytosine-containing or cytidine-containing compound, creatine-containing compound, adenosine-containing, adenosine-elevating compound, omega-3 fatty acids, or combinations thereof to a mammal.

Another prior art U.S. Pat. No. 5,482,706 A assigned to Yasutaka Igari, et. al, entitled “Transmucosal therapeutic composition” discloses a transmucosal therapeutic composition comprising a physiologically active peptide or protein and a cytidine nucleotide derivative. The above composition results in a satisfactory transmucosal absorption of physiologically active peptides or proteins that are otherwise hardly absorbed from the mucosa. Since it allows self-administration to the mucosa, such as the nasal, vaginal or digestive tract mucosa, instead of injection which causes pain, the invention is of great utility as a pharmaceutical dosage form for physiologically active peptides or proteins which must be administered over a protracted time.

Though the above-mentioned prior arts disclose various methods for treating neurodegenerative diseases and other conditions, none of them disclose a method of treating patients with bone-related diseases or to accelerate the healing following fractures and other disorders.

Therefore, there is a need for a method of treating or preventing bone-related diseases and a variety of disorders and other conditions that accelerates healing using an effective amount of citicoline alone or in combination with other potential nutraceutical agents. Also, there is a need for a method of treating a human using a citicoline with or without other nutraceutical agents configured to promote and accelerate delayed union and non-union of fractures.

### SUMMARY

The present invention generally discloses a method of treating or preventing various bone-related diseases. Also, the present invention discloses a method for treating or preventing various bone-related diseases and disorders in humans using a citicoline alone or in combination with other potential nutraceutical agents. Further, the present invention discloses a method of treating a human using a citicoline with or without other nutraceutical agents configured to promote fracture healing and accelerate healing in delayed union and non-union of fractures.

In one embodiment, the method of treating various bone-related diseases and disorders in humans. In one embodiment, the method of treatment involves the administration of an effective amount of citicoline (Cytidine Diphosphate choline or Cytidine 5′-diphosphocholine) alone or in combination with other nutraceutical agents like vitamin C. The citicoline provides effective clinical benefits to humans in need of fracture healing treatment. In one embodiment, the bone-related disease is osteoporosis. In one embodiment, the effective amount of citicoline is administrated to the human in need of such treatment. In one embodiment, the administration of the effective amount of citicoline is topical. In one embodiment, the administration of the effective amount of citicoline is oral. In one embodiment, the effective amount of citicoline is injected into the human body to promote tissue repair.

In one embodiment, the method of treating patients using an effective amount of citicoline with or without other nutraceutical agents accelerates fracture healing. In one embodiment, the method of treating patients using an effective amount of citicoline with or without other nutraceutical agents promotes healing in delayed union and non-union. In one embodiment, the method of treating patients using an effective amount of citicoline with or without other nutraceutical agents promotes healing in infected non-union of fractures. In one embodiment, the method of treating patients using an effective amount of citicoline with or without other nutraceutical agents speeds up the fracture healing. In one embodiment, the method of treating patients using an effective amount of citicoline with or without other nutraceutical agents speeds up osteoporotic fracture healing. In one embodiment, the method of treating patients using an effective amount of citicoline also promotes spinal cord regeneration in spinal cord injury in combination with bone marrow aspirate.

The above summary contains simplifications, generalizations, and omissions of detail and is not intended as a comprehensive description of the claimed subject matter but, rather, is intended to provide a brief overview of some of the functionality associated therewith. Other systems, methods, functionality, features, and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 shows a graph of real time PCR amplification of muscarinic M1 receptor mRNA in the T9-T12 region of spinal cord of control and experimental groups of rats.

FIG. 2 shows a confocal image of muscarinic M1 receptors.

FIG. 3 shows a confocal image of TOPRO 3 staining.

### DETAILED DESCRIPTION OF EXAMPLE EMBODIMENTS

A description of embodiments of the present invention will now be given with reference to the Figures. It is expected that the present invention may be embodied in other specific forms without departing from its spirit or essential characteristics. The described embodiments are to be considered in all respects only as illustrative and not restrictive.

In one embodiment, a method of treating or preventing various bone-related diseases and disorders in humans or patients is disclosed. In one embodiment, the method of treatment treats or prevents various bone-related diseases and other disorders and conditions in humans using citicoline alone or in combination with other potential nutraceutical agents like vitamin C. In one embodiment, the method of treating the human affected with fractures and bone recovery uses citicoline with or without other nutraceutical agents.

In one embodiment, the method administrates an effective amount of citicoline (Cytidine Diphosphate choline or Cytidine 5′-diphosphocholine) alone or in combination with other potential nutraceutical agents like vitamin C to humans in need of such treatments. Citicoline with or without other nutraceutical agents provide effective clinical benefits to humans in need of fracture healing treatment. In one embodiment, the bone-related disease is osteoporosis. In one embodiment, the administration of the effective amount of citicoline is topical. In one embodiment, the administration of the effective amount of citicoline is oral. In one embodiment, the effective amount of citicoline is injected into the human body to promote tissue repair.

In one embodiment, the method of treating patients using an effective amount of citicoline with or without other nutraceutical agents accelerates fracture healing. In one embodiment, the method of treating patients using an effective amount of citicoline with or without other nutraceutical agents promotes healing in delayed union and non-union of fractures. In one embodiment, the method of treating patients using an effective amount of citicoline with or without other nutraceutical agents promotes healing in infected non-union of fractures. In one embodiment, the method of treating patients using an effective amount of citicoline with or without other nutraceutical agents speeds up the fracture healing. In one embodiment, the method of treating patients using an effective amount of citicoline with or without other nutraceutical agents speeds up osteoporotic fracture healing. In one embodiment, the method of treating patients using an effective amount of citicoline promotes spinal cord regeneration in spinal cord injury in combination with bone marrow aspirate.

### EXAMPLES

### Example 1

Spinal cord injury results in disruption of brain-spinal cord fiber connectivity, leading to progressive tissue damage at the site of injury and resultant paralysis of varying degrees. The current study investigated the role of autologous bone marrow modulated with neurotransmitters and neurotransmitter stimulating agent, Citicoline, in the spinal cord of spinal cord injured rats.

### Example 2—Methods

Radioreceptor assay using [3H] ligand was carried out to quantify muscarinic receptors. Gene expression studies were done using real time PCR analysis.

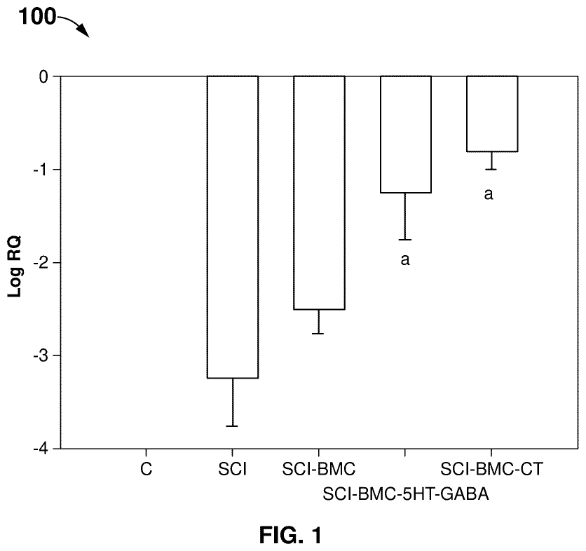

Referring to FIG. 1, a graph 100 of real time PCR Amplification of muscarinic M1 receptor mRNA in the T9-T12 region of spinal cord of control and experimental groups of rats. The values are mean±S.E.M. of 4-6 separate experiments. Each group consists of 8-10 rats. a p<0.001 when compared to control. Abbreviations used: C: Control; SCI: Spinal Cord Injury; SCI+BMC: Spinal Cord injury+Bone Marrow; SCI+BMC+5HT+GABA: Spinal Cord Injury+Bone Marrow+Serotonin+GABA; SCI+BMC+CT: Spinal Cord injury+Bone Marrow+Citicoline; a: p<0.001; b: p<0.01 when compared to control; d: p<0.001; e: p<0.01 when compared to spinal cord injured.

### Example 3—Results

Scatchard analysis of muscarinic M1 receptor showed significantly decreased Bmax (p<0.001) and Kd (p<0.01) compared to control and significant reversal (p<0.001) in both the treatment groups (spinal cord injury treated with 5HT and GABA, and spinal cord injury treated with Citicoline). Muscarinic M1 receptor gene expression in spinal cord injured group showed significant down regulation (p<0.001) compared to control, and both the treatment groups significantly reversed (p<0.001) these changes to near control when compared to spinal cord injured group. The confocal microscopic study using specific antibody of muscarinic M1 confirmed the gene expression studies.

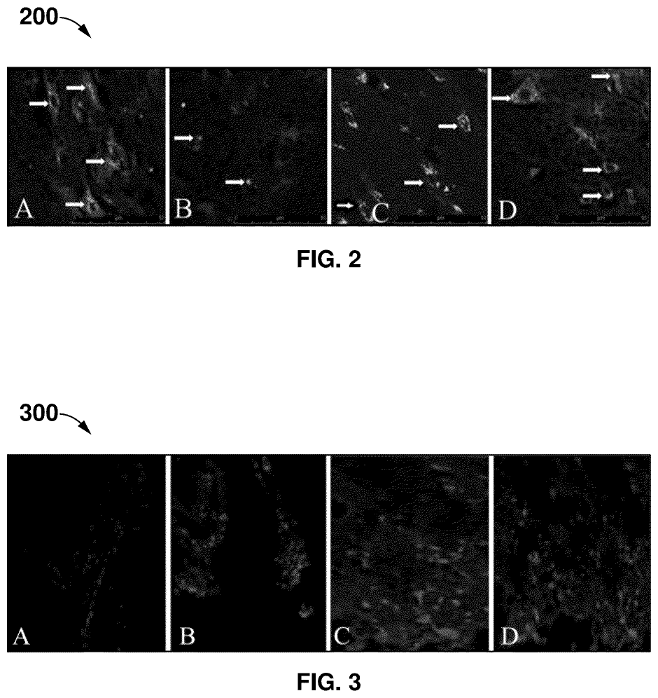

Referring to FIG. 2, a confocal image 200 of muscarinic M1 receptors of control (A), spinal cord injury (B), spinal cord injury+bone marrow+5HT+GABA (C), and spinal cord injury+bone marrow cell+citicoline (D) rats using immunofluorescent microscope with muscarinic M1 receptor specific primary antibody and FITC labeled as secondary antibody. Arrows in white shows muscarinic M1 receptors. Scale bar=50 μm. The arrows depict neuronal visualisation in treatment groups similar to the control which is not seen in injured group.

Referring to FIG. 3, a confocal image 300 of TOPRO 3 staining of control (A), spinal cord injury (B), spinal cord injury+bone marrow+5HT+GABA, (C) and spinal cord injury+bone marrow 1+citicoline, (D) scale bar=50 μm.

### Example 4—Conclusion

Thus, our results suggest that the neurotransmitters combination along with bone marrow or Citicoline with bone marrow can reverse the muscarinic receptor alterations in the spinal cord of spinal cord injured rats, which is a promising step towards a better therapeutic intervention for spinal cord injury because of the positive role of cholinergic system in regulation of both locomotor activity and synaptic plasticity.

Advantageously, the method of treatment administrates an effective amount of citicoline to humans for treating or preventing various bone-related diseases and disorders. The citicoline with or without other nutraceutical agents provides effective clinical benefits to humans in need of fracture healing treatment. The method of treatment is used for the acceleration of fracture healing. Also, the method of treatment is used for the promotion of fracture healing. Further, the method of treatment is used for speeding up of fracture healing.

Although a single embodiment of the invention has been illustrated in the accompanying drawings and described in the above detailed description, it will be understood that the invention is not limited to the embodiment developed herein, but is capable of numerous rearrangements, modifications, substitutions of parts and elements without departing from the spirit and scope of the invention.

The foregoing description comprises illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

What is claimed is:

1. A method of treating or preventing various bone-related diseases and disorders in a human consisting of, administering an effective amount of citicoline (Cytidine Diphosphate choline or Cytidine 5′-diphosphocholine) alone or in combination with other potential nutraceutical agents like vitamin C.

2. The method of claim 1, accelerates fracture healing.

3. The method of claim 1, accelerates osteoporotic fracture healing.

4. The method of claim 1, promotes healing in delayed union and non-union of fractures.

5. The method of claim 1, promotes healing in infected non-union of fractures.

6. The method of claim 1, promotes spinal cord regeneration in spinal cord injury in combination with bone marrow aspirate.

7. The method of claim 1, wherein the bone-related disease is osteoporosis.

8. The method of claim 1, wherein the effective amount of citicoline with or without other nutraceutical agents is administrated to the human in need of such treatment.

9. The method of claim 1, wherein the administration of the effective amount of citicoline is topical.

10. The method of claim 1, wherein the administration of the effective amount of citicoline is oral.

11. The method of claim 1, wherein the effective amount of citicoline is injected into the human body to promote tissue repair.
